(19) United States
(12) Patent Application Publication
(10) Pub. No.: US 2004/0092955 A1
(43) Pub. Date: May 13, 2004

(54) Umbilical cord closure device

(76) Inventors: Swee Hoe Phua, Singapore (SG); Wai Chiau Kwok, Singapore (SG); Soei Kiat Yap, Singapore (SG)

Correspondence Address: OSTROLENK FABER GERB & SOFFEN, 1180 AVENUE OF THE AMERICAS, NEW YORK, NY, 100368403

(21) Appl. No.: 10/332,084

(22) PCT Filed: Feb. 23, 2001
(86) PCT No.: PCT/SG01/00026

(30) Foreign Application Priority Data
Jul. 6, 2000 (WO) PCT/SG00/00097

(51) Int. Cl. A61B 17/42

(52) U.S. Cl. 606/120

(57) ABSTRACT

An umbilical cord closure device comprising an elongate strap (20) connected at one end of to fastening means (10), the strap (20) and fastening means (10) having co-operative means for engagement to form a tightenable loop for receiving an umbilical cord.

### BACKGROUND AND FIELD OF THE INVENTION

This invention relates to an umbilical cord closure device.

The umbilical cord connects a baby to his/her mother for supply of nutrients during pregnancy. At birth, it is necessary for the umbilical cord to be detached. Conventionally, this has been done by applying two umbilical cord clamps at the mother (placenta) and child ends of the umbilical cord and severing the cord between the clamps. In time the severed ends of the cord will fall off together with the clamps. Umbilical cord clamps generally act like a pair of tongs having first and second clamping surfaces pivoting at a hinge with the umbilical cord being clamped closed between the clamping surfaces. It is a disadvantage of such prior art umbilical cord clamps that they have difficulty in accommodating umbilical cords of differing diameter. Furthermore, such clamps tend to be large, heavy and expensive.

It is an object of the invention to provide a novel umbilical cord closure device which alleviates at least one of the disadvantages of the prior art.

### SUMMARY OF THE INVENTION

According to the invention, there is provided an umbilical cord closure device comprising an elongate strap connected at one end to a fastening means, the strap and fastening means having co-operative means for engagement to form a tightenable loop for receiving a said umbilical cord.

### BRIEF DESCRIPTION OF THE DRAWINGS

Embodiments of the invention will now be described, by way of example, with reference to the accompanying drawings in which

FIG. 1 is a three dimensional view of a first embodiment of the invention;

FIG. 2 is an enlarged view of a fastener of the embodiment of FIG. 1; and

FIG. 3 is a cross sectional view through A-A of FIG. 2 showing the strap connected to the fastener.

FIG. 4 is a view similar to FIG. 2 showing a second embodiment of the invention in which a cover to hide the free end of the strap is provided.

FIG. 5 is a view similar to FIG. 4 showing the cover closed.

FIG. 6 is a front view of the third embodiment of the invention;

FIG. 7 is a view in the direction of arrow A of FIG. 6;

FIG. 8 is a view similar to FIG. 6 with the strap engaged with the fastener to form a loop;

FIG. 9 is a three dimensional view of the loop of FIG. 8;

FIG. 10 is an enlarged view of the portion labelled B of FIG. 6.

### DETAILED DESCRIPTION OF THE PREFERRED EMBODIMENTS

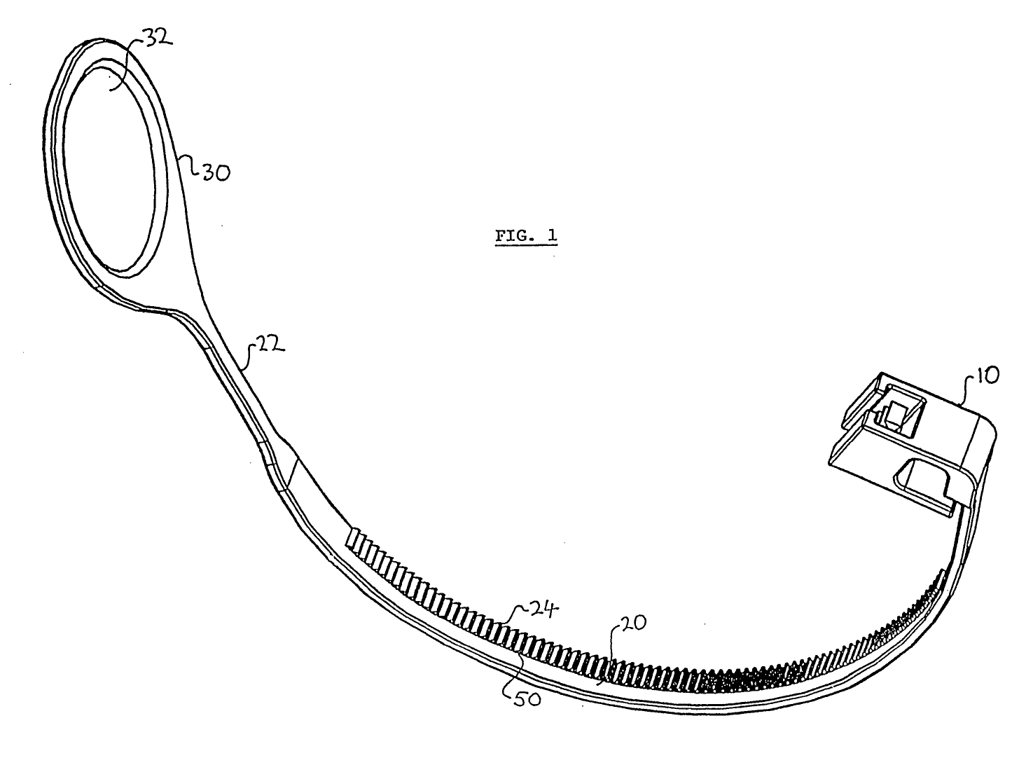

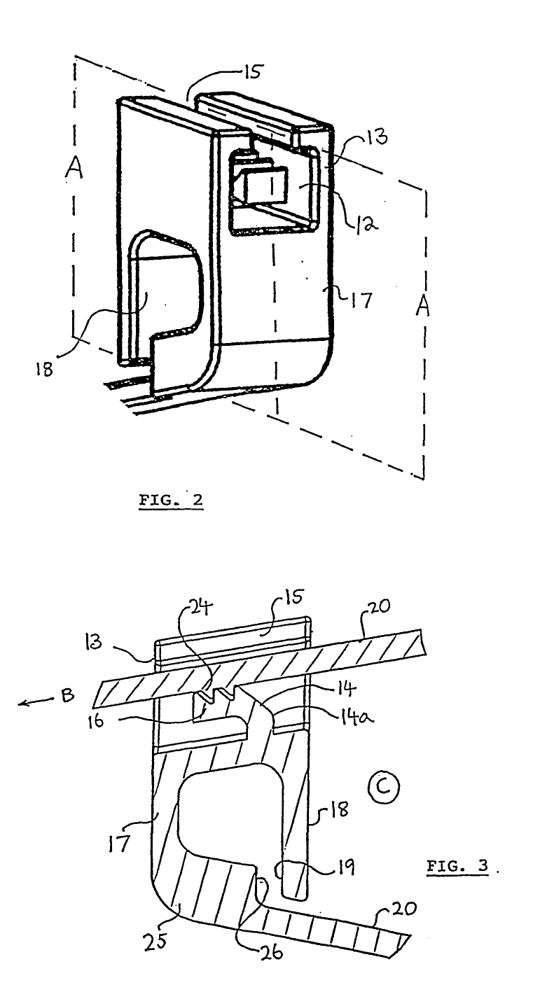

With reference to FIGS. 1-3, an umbilical cord closure device being a first embodiment of the invention is illustrated. The closure device is formed as one piece from injection moulded plastics material for example nylon. The device comprises a strap 20 connected at one end to a fastener 10 and at the other end to a handle 30. The strap 20 is connected to the handle 30 via a region 22 of narrower cross section. As best illustrated in FIG. 2, the fastener 10 is provided with a cavity 12 in which a locking pawl 14 is disposed. The cavity is closed on three sides but on the fourth side is provided with a slot 15. The slot 15 is of a width sufficient just to receive the narrower portion 22 of strap 20 but not the main portion 20. The cavity 12 is of a width sufficient slidably to receive the main portion 20. The strap 20 is provided with a elongate array of teeth 24 which are adapted to engage corresponding teeth 16 of the pawl 14 as shown in FIG. 3. The pawl 14 has a resilient support 14a which deflects to allow the strap to be pulled through the cavity, with the teeth 16, 24 engage, in a click-stop manner.

The device is preferably formed with the first and second sides of the mold running along an edge 50 of the array of teeth 24 so that any flashing at the parting line of the mold is not formed on surfaces which are likely to engage the umbilical cord of the child.

The fastener 10 is connected to the strap 20 by means of a thin bridging portion 17. Parallel to the thin bridging portion a reaction member 18 is provided which extends from the fastener 10 almost to reach the strap 20. Connecting the strap 20 to the bridging portion 17 is a region 25 which includes an abutment surface 26 against which the free end 19 of the reaction member 18 can engage.

In use, hospital staff can attach the embodiment of the invention to an umbilical cord by grasping handle 30 (preferably by putting a finger through opening 32) and passing strap portion 22 through opening 15 in fastener 10 and then pulling strap 20 through cavity 12. This will cause teeth 24 of strap 20 to engage teeth 16 of pawl 14 in a click-stop manner. The teeth of both pawl 14 and strap 20 are formed to allow one-way movement only in the direction of arrow B by having a leading slanted surface and a trailing perpendicular surface as shown in FIG. 3. Pulling the strap 20 through fastener 10 will thus cause a loop to be formed which can be tightened. As the loop is tightened the umbilical cord will be forced into a region C of FIG. 3 between the strap 20 and reaction member 18. The reaction member 18 is arranged to be non-resilient. This might, however, flex slightly when contacted by the umbilical cord but only until abutment of surfaces 19, 26. The arrangement of strap 20 and member 18 thus provides solid surfaces between which the umbilical cord may be pinched and thus closed off. At that point the handle 30 together with any excess portion of the strap 20 can be cut along face 13 leaving a neat closure.

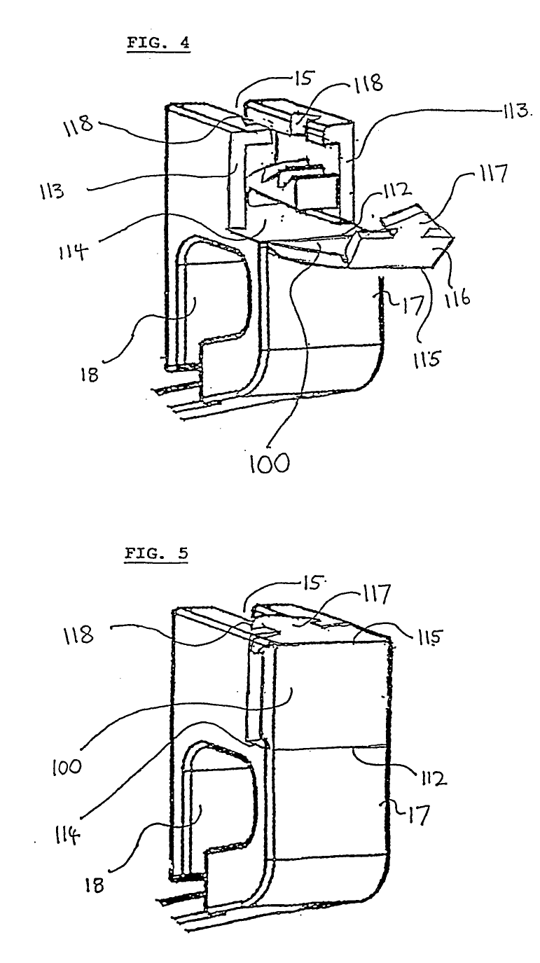

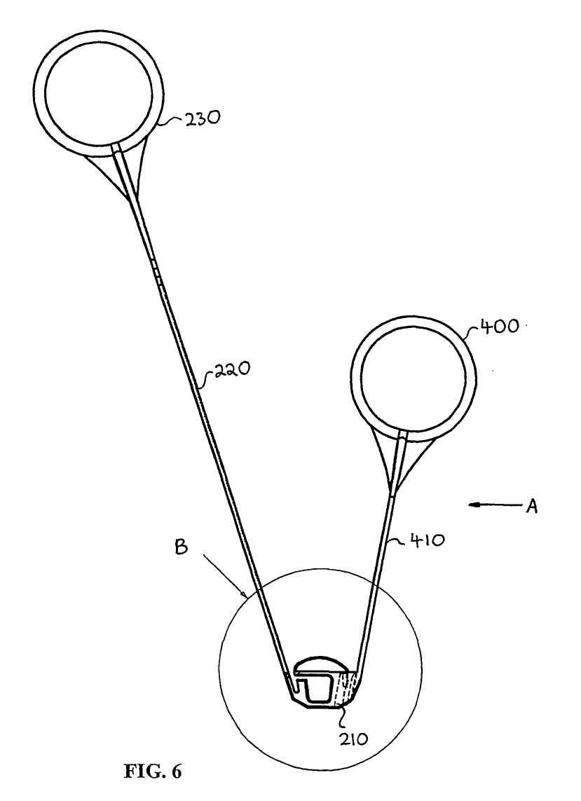

A second embodiment of the invention is shown in FIGS. 4 and 5. This embodiment differs from the previous embodiment by the addition of a cover 100 to the fastener 10. The sides of cavity 12 do not extend the length of the fastener 10, but are cut back to form a ledge 114 and recessed surface 113. The cover 100 is connected to an edge of ledge 114 via a film hinge 112. The cover 100 is provided with a fastener 116 connected via film hinge 115 and having an arrow-shaped portion 117 adapted to be press-fitted into a corresponding arrow-shaped slit 118 in the cavity top surface. In use, once the strap 20 has been cut along face 113 in a similar manner to that described in the preceding paragraph, the cover 100 is moved from the position shown in FIG. 4 to that shown in FIG. 5, with the portion 117 engaged with slit 118 so that the cover 100 hides the cut end of strap 20 to prevent any sharp edge of the cut end from being exposed.

Occasionally during post-natal procedure, it is necessary to remove the closure device. This can be easily accomplished with the described embodiments of the present invention by cutting through the bridge portion 17.

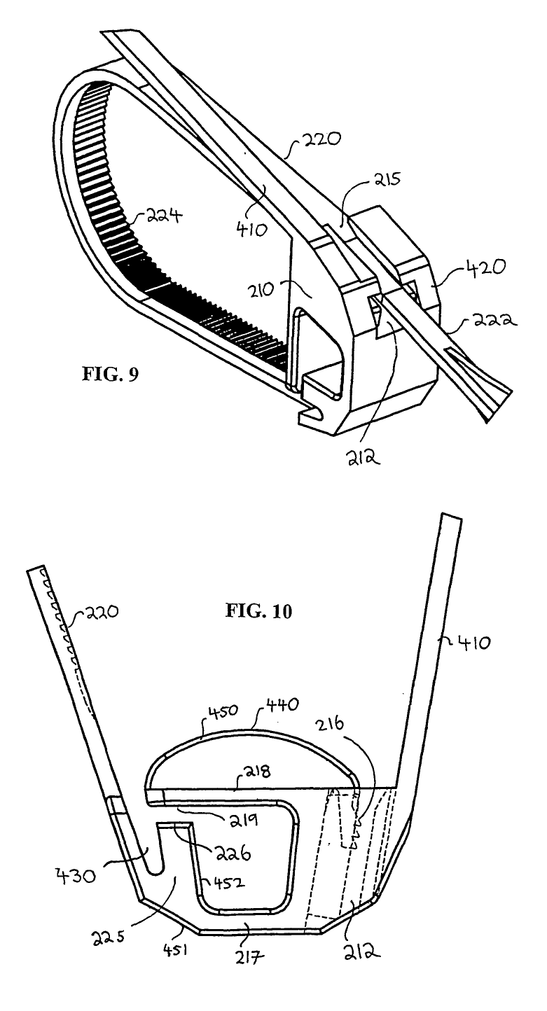

FIGS. 6-10 illustrate a third embodiment of the invention which is broadly similar in concept to the embodiment of FIGS. 1-3 with similar components being identified by similar reference numerals with the addition of 200. As in the first embodiment, the umbilical cord closure device has a strap 220 connected at one end to a fastener 210 and at the other to a handle 230 via a region of narrower cross-section 222. The strap 220 and fastener 210 together form a loop, in the manner of the first embodiment, as best shown in FIG. 9.

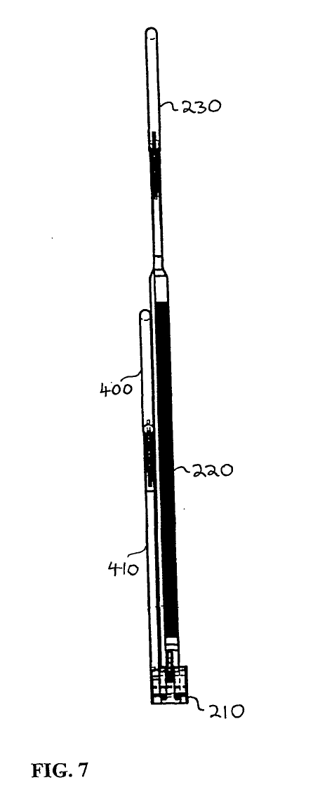

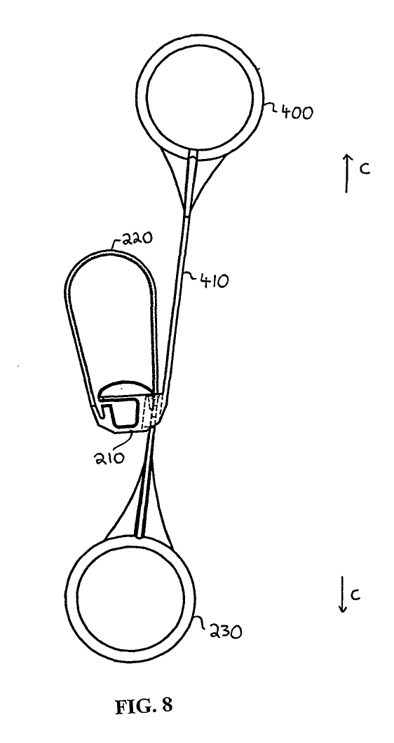

The third embodiment includes an additional handle 400 connected to fastener 210 via strap 410. As best shown in FIGS. 7 and 9 the strap 410 is connected to fastener 210 so as to be offset relative to strap 220 and to the side of slot 215. The strap 220 is also slightly offset relative to the centre line of fastener 210 so that the straps 220, 410 do not interfere with one another when the loop is tightened. The function of additional handle/strap 400/410 is to allow the user to gain a greater purchase when tightening the closure device, by being able to grasp both handles and pull them apart, in the direction of arrows C of FIG. 8 to tighten the loop.

The fastener 210 is further provided with a chamfered surface 420 at one end of cavity 212. The chamfered surface allows the strap 222 to be cut at an angle, flush to the surface 420 once the loop has been tightened thus reducing the risk of sharp edges once cut.

In order to increase the ability of the strap 220 and fastener 210 to mate against one another to close an umbilical cord, region 225 which connects strap 220 to bridging portion 217 includes a gap 430 which allows the strap 220 to be pulled closer to the reaction member 218 when being tightened. The reaction member 218 is further provided with an arcuate umbilical cord engaging surface 440 which follows the line of the strap 220 when this is tightened.

As best shown in FIG. 10, the edge surfaces of the fastener 210 are also chamfered along surfaces 450, 451, 452, to reduce the risk of cuts. A cover in the manner of the embodiment of FIGS. 4 and 5 may be provided.

1. An umbilical cord closure device comprising an elongate strap connected to fastening means, the strap and fastening means having co-operative means for engagement to form a tightenable loop for receiving a said umbilical cord.

2. A device as claimed in claim 1 wherein a handle is connected to the strap.

3. A device as claimed in claim 2 wherein the handle is provided with a finger hole.

4. A device as claimed in claim 2 or claim 3 further comprising a further handle connected to the fastening means.

5. A device as claimed in claim 4 wherein the handle is connected to the fastening means by means of a further strap.

6. A device as claimed in claim 5 wherein the straps are connected to the fastening means in parallel.

7. A device as claimed in any one of claims 4-6 wherein the further handle is provided with a finger hole.

8. A device as claimed in any one of the preceding claims wherein the fastening means includes a cavity having an opening arranged to receive the strap.

9. A device as claimed in claim 8 wherein the strap includes a first region of reduced width receivable by a slot in the cavity and a second region of enlarged width receivable by the cavity but not receivable by the slot.

10. A device as claimed in claim 8 or claim 9 wherein the cavity has a further opening through which the strap is arranged to pass, the further opening having a chamfered surface.

11. A device as claimed in any one of the preceding claims wherein the co-operating engagement means comprises a plurality of teeth formed on the strap and a corresponding toothed pawl engageable with teeth of the strap.

12. A device as claimed in any one of the preceding claims wherein the fastening means is connected to the strap via a severable bridge portion.

13. A device as claimed in any one of the preceding claims further comprising a reaction member extending from the fastening means, the reaction member being arranged to face inwardly of the loop when formed.

14. A device as claimed in claim 13 wherein a surface of the reaction member arranged to face inwardly of the loop when formed is arcuate.

15. A device as claimed in claim 13 wherein the strap forming part of the co-operative means is connected to the fastening means via a connecting portion, the connecting portion having an abutment surface arranged to engage the reaction member when the loop is tightened.

16. A device as claimed in claim 15 wherein the strap is connected to the connection portion at a position spaced from the abutment surface.

17. A device as claimed in any one of the preceding claims wherein the device is formed by injection molding.

18. A device as claimed in any one of the preceding claims wherein the fastening means includes a cover adapted to cover a free end of the strap.

19. An umbilical cord closure device comprising an elongate strap connected at one end to a fastener and securable at a selectable further point to the fastener, the fastener and strap being provided with co-operable engagement means for selective engagement at said point; the arrangement being such that the strap is connectable to the fastener to form a loop tightly to receive a said umbilical cord.

20. A device as claimed in claim 19 wherein the strap is provided with a handle and further comprising a further handle connected to the fastener.
